(19) United States
(12) Patent Application Publication
(10) Pub. No.: US 2020/0342598 A1
(43) Pub. Date: Oct. 29, 2020

(54) IMAGE DIAGNOSIS SUPPORT SYSTEM AND IMAGE DIAGNOSIS SUPPORT METHOD

(71) Applicant: OLYMPUS CORPORATION, Tokyo (JP)

(72) Inventor: Fumiyuki SHIRATANI, Tokyo (JP)

(73) Assignee: OLYMPUS CORPORATION, Tokyo (JP)

(21) Appl. No.: 16/928,416

(22) Filed: Jul. 14, 2020

Related U.S. Application Data
(63) Continuation of application No. PCT/JP2018/001053, filed on Jan. 16, 2018.

(51) Int. Cl. G06T 7/00; G06T 7/11; G06T 5/00; G06T 5/20; A61B 5/00; A61B 1/04

(52) U.S. Cl. CPC G06T 7/0012; G06T 7/11; G06T 5/003; G06T 5/20; A61B 5/7267; G06T 2207/30096; A61B 1/04; G06T 2207/20201; G06T 2207/10068; G06T 2207/30168; A61B 5/4887

(57) ABSTRACT

An image diagnosis support system includes: an input unit that receives an input of an image; a specifying unit that specifies a specular reflection region and a non-specular reflection region in a region of interest in the image; and a determination unit that determines whether the region of interest is an inadequate region that is inadequate for diagnosis on the basis of an image processing result for at least one of the specular reflection region and the non-specular reflection region.

### CROSS-REFERENCE TO RELATED APPLICATIONS

This application is based upon and claims the benefit of priority from International Application No. PCT/JP2018/001053, filed on Jan. 16, 2018, the entire contents of which is incorporated herein by reference.

### BACKGROUND OF THE INVENTION

### 1. Field of the Invention

The present invention relates to an image diagnosis support system and an image diagnosis support method.

### 2. Description of the Related Art

There are known devices that support the diagnosis of endoscopic images. There has been a conventionally proposed technique of excluding an endoscopic image from a processing target in a case where the endoscopic image includes a blur (for example, patent document 1).

However, in consideration of the local occurrence of blurs and shakes in an endoscope, there might be cases where regions of interest in terms of diagnosis include no blurs or shakes even when the endoscopic image includes a blur or a shake. In this case, at least the region of interest should be determined as a diagnosis target.

### SUMMARY OF THE INVENTION

The present invention has been made in view of such circumstances and aims to provide an image diagnosis support technology capable of suitably determining a diagnosis target.

In order to solve the above problem, an image diagnosis support system according to an aspect of the present invention includes a processor that includes hardware, wherein the processor is configured to receive an input of an image; specify a specular reflection region and a non-specular reflection region in a region of interest in the image; and determine whether the region of interest is an inadequate region that is inadequate for diagnosis on the basis of an image processing result for at least one of the specular reflection region and the non-specular reflection region.

Another aspect of the present invention is an image diagnosis support method. This method includes: receiving an input of an image; and determining whether a region of interest is an inadequate region that is inadequate for diagnosis on the basis of an image processing result for at least one of the specular reflection region and the non-specular reflection region in the region of interest in the image.

Note that any combination of the above constituent elements, and representations of the present invention converted between a method, a device, a system, a recording medium, a computer program, or the like, are also effective as an aspect of the present invention.

### BRIEF DESCRIPTION OF THE DRAWINGS

Embodiments will now be described, byway of example only, with reference to the accompanying drawings that are meant to be exemplary, not limiting, and wherein like elements are numbered alike in several figures, in which:

FIG. 1 is a block diagram illustrating functions and configurations of an image diagnosis support system according to a first embodiment;

FIG. 2 is a diagram illustrating a schematic configuration of a Faster R-CNN;

FIG. 3 is a diagram illustrating a learning procedure of the Faster R-CNN;

FIG. 4 is a diagram illustrating a schematic processing configuration of a CNN;

FIG. 5 is a flowchart illustrating an example of a series of processes in the image diagnosis support system according to the first embodiment;

FIG. 6 is a block diagram illustrating functions and configurations of an image diagnosis support system according to a second embodiment;

FIG. 7 is a flowchart illustrating an example of a series of processes in the image diagnosis support system according to the second embodiment;

FIG. 8 is a block diagram illustrating functions and configurations of an image diagnosis support system according to a third embodiment;

FIG. 9 is a flowchart illustrating an example of a series of processes in the image diagnosis support system according to the third embodiment.

### DETAILED DESCRIPTION OF THE INVENTION

The invention will now be described by reference to the preferred embodiments. This does not intend to limit the scope of the present invention, but to exemplify the invention.

Hereinafter, the present invention will be described based on preferred embodiments with reference to the drawings.

### First Embodiment

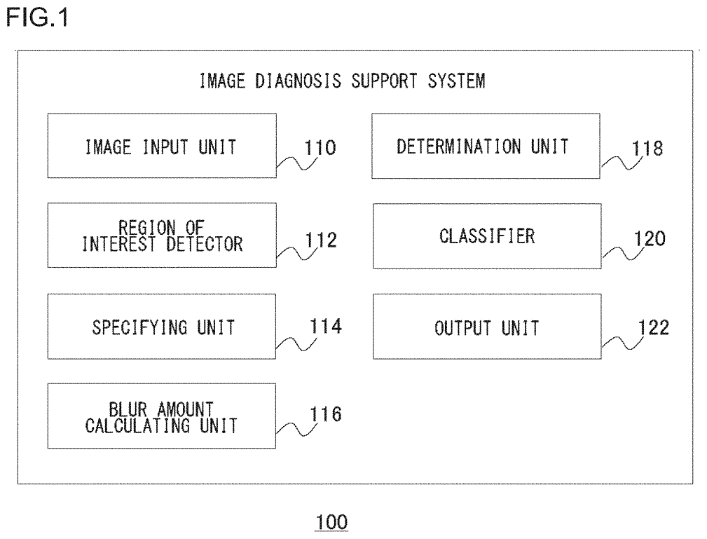

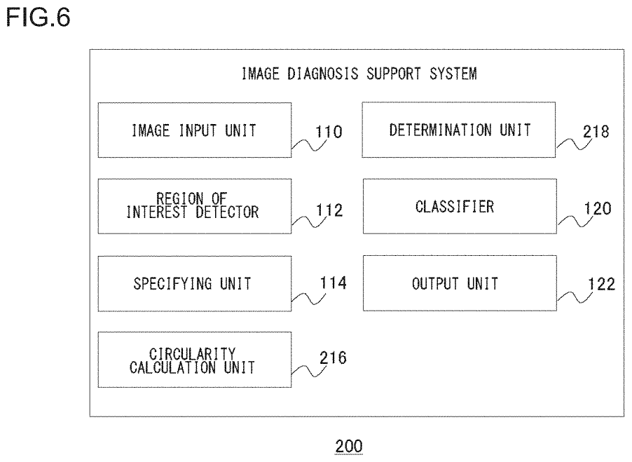

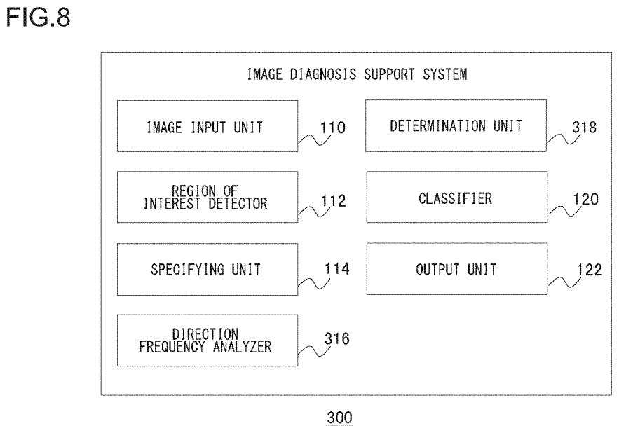

FIG. 1 is a block diagram illustrating functions and configurations of an image diagnosis support system 100 according to a first embodiment. Each of blocks illustrated here can be implemented by elements or mechanical devices such as a central processing unit (CPU) of a computer in terms of hardware and can be implemented by a computer program in terms of software. However, functional blocks implemented by cooperation of hardware and software are depicted here. Accordingly, implementability of these functional blocks in various forms using the combination of hardware and software would be understandable by those skilled in the art. The similar applies to FIGS. 6 and 8 described below.

The image diagnosis support system 100 supports diagnosis of a lesion using an endoscopic image. The endoscopic image is captured by a conventional endoscope in which a scope is inserted into the body, or by a capsule endoscope.

The image diagnosis support system 100 includes an image input unit 110, a region of interest detector 112, a specifying unit 114, a blur amount calculation unit 116, a determination unit 118, a classifier 120, and an output unit 122.

The image input unit 110 receives an input of an endoscopic image from a user or another device. The region of interest detector 112 performs a detection process of detecting a region of interest being a lesion candidate region, on the endoscopic image received by the image input unit 110. Depending on the endoscopic image, there are cases where no region of interest is detected, or one or more regions of interest are detected. The region of interest detector 112 executes a region of interest detection process using a convolutional neural network (CNN). This will be described below.

In a case where the region of interest detector 112 detects a region of interest in the endoscopic image, the specifying unit 114 specifies a specular reflection region and a non-specular reflection region in the region of interest. Note that an endoscope has a special characteristic that the frequency of occurrence of specular reflection is relatively high because of positional proximity of the light source, the subject, and the light receiving element, in typical cases. Specifically, the specifying unit 114 specifies, in the region of interest, a pixel or a group of pixels having a pixel value representing brightness that is a predetermined threshold or more as a specular reflection region and specifies pixels or a group of pixels having a pixel value less than the predetermined value as a non-specular reflection region. At this time, the specifying unit 114 may perform dilation/erosion processing as needed.

The blur amount calculation unit 116 calculates the blur amount in the non-specular reflection region. In a case where there is a plurality of regions of interest, the blur amount calculation unit 116 calculates the blur amount of the non-specular reflection region for each of regions of interest.

The blur amount calculation unit 116 first extracts an edge (that is, a luminance change point) from the non-specular reflection region. Note that a known technique such as Canny Edge Detector may be used to extract the edge.

Subsequently, the blur amount calculation unit 116 calculates the blur amount of each of pixels of the extracted edge. A known method can be used to calculate the blur amount. The blur amount calculation unit 116 of the present embodiment calculates the blur amount using the method described in non-patent document 1. That is, the blur amount calculation unit 116 calculates a blur amount (σ) by the following Formula (1).

$$\begin{matrix}Formula ( 1 ) & \\ σ = \frac{1}{\sqrt{{R}^{2} - 1}} {σ}_{0} & Formula ( 1 )\end{matrix}$$

Here

σ₀: Standard deviation of the Gaussian kernel that represents a small amount of blur added by applying a Gaussian filter

R: Maximum value of a ratio of edge gradient before and after adding a small amount of blur.

This technique can be understood as a method using a difference that the gradient of the edge becomes less steep more significantly at a time of adding a small amount of blur in a case where the edge gradient is relatively steep, that is, the pixel is relatively unblurred, and this leads to a large maximum value of the ratio of the edge gradient before and after the addition of blur, whereas the gradient of the edge becomes less steep less significantly at a time of adding a small amount of blur in a case where the edge gradient is relatively gentle, that is, the pixel is relatively blurred, and this leads to a small maximum value of the ratio of the edge gradient before and after the addition of the blur.

The blur amount calculation unit 116 further calculates a mean value of the calculated blur amounts of each of the pixels and sets the mean value as a blur amount of the non-specular reflection region.

In the present embodiment, the determination unit 118 determines whether the region of interest is a diagnostically inadequate region with a blur on the basis of the image processing result for the non-specular reflection region of the region of interest, that is, on the basis of the blur amount of the non-specular reflection region. In a case where there is a plurality of regions of interest, the determination unit 118 determines whether the region of interest is a diagnostically inadequate region with a blur for each of regions.

Specifically, the determination unit 118 determines whether the blur amount calculated by the blur amount calculation unit 116 is larger than a threshold Th1. In a case where the blur amount is larger than the threshold Th1, the determination unit 118 determines that the region of interest is blurred, that is, the region of interest is a diagnostically inadequate region. In a case where the blur amount is the threshold Th1 or less, the determination unit 118 determines that the region of interest is not blurred, that is, the region of interest is not the diagnostically inadequate region.

The classifier 120 performs a classification process of classifying (discriminating) whether the lesion indicated by the region of interest in the endoscopic image is benign or malignant. The classifier 120 according to the present embodiment executes the classification process in a case where the determination unit 118 determines that the region of interest is not a diagnostically inadequate region, while the classifier 120 does not execute the classification process in a case where the determination unit 118 determines that the region of interest is a diagnostically inadequate region. The classifier 120 executes a classification process using a convolutional neural network. This will be described below.

The output unit 122 outputs the processing result of the classifier 120 to a display, for example. When the region of interest is not a diagnostically inadequate region and the classification process of the region of interest has been executed by the classifier 120, the output unit 122 outputs the result of the classification process, that is, the result of classification (discrimination) indicating whether the lesion indicated by the region of interest is benign or malignant. In another case where the region of interest is determined as a diagnostically inadequate region and the classification process by the classifier 120 has not been executed, the output unit 122 outputs indication that the region of interest is a diagnostically inadequate region.

Note that the classifier 120 may execute the classification process regardless of the determination result by the determination unit 118, that is, regardless of whether the region of interest is a diagnostically inadequate region, and the output unit 122 may output the classification result in a case where the region of interest is not a diagnostically inadequate region.

The above is the basic configuration of the image diagnosis support system 100.

Next, a region of interest detection process performed using a CNN will be described. Here, a case where the lesion is a polyp will be described. A detection CNN is trained beforehand using a polyp image and a normal image. After the training, an image is input to the detection CNN, and then, a polyp candidate region is detected. In a case where no candidate region is detected in the image, the image is determined as a normal image.

Hereinafter, a case where Faster R-CNN is used as the detection CNN will be described. The Faster R-CNN includes two CNNs, a Region Proposal Network (RPN) that detects candidate frames (rectangles) from an image and a Fast R-CNN (FRCNN) that examines whether candidate frames are detection targets. By sharing the feature extraction CNN, both CNNs realize a high-speed detection process.

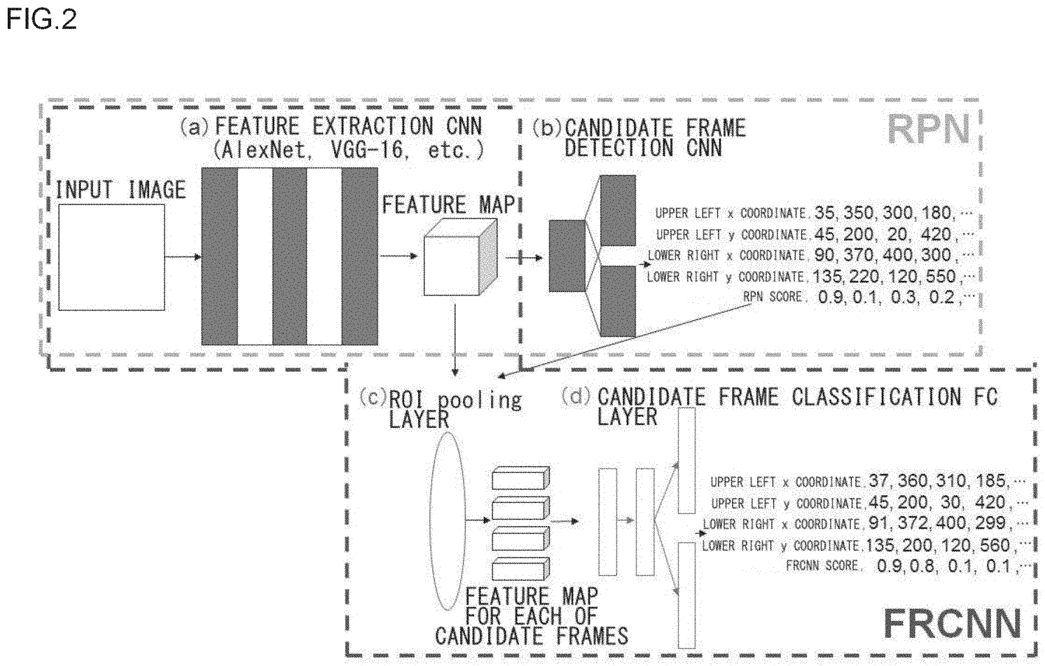

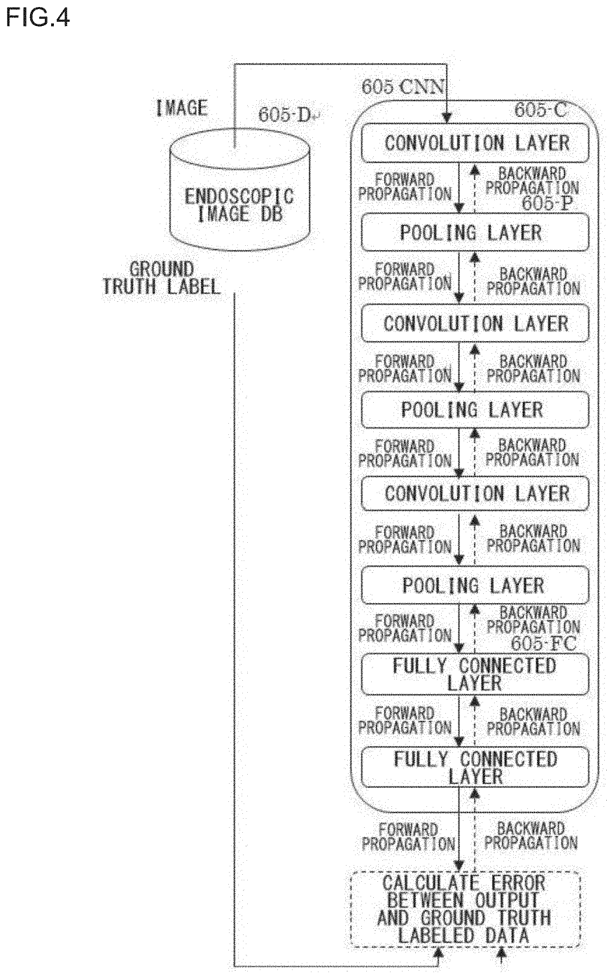

FIG. 2 illustrates a schematic configuration diagram of a Faster R-CNN. First, an image is input to the feature extraction CNN illustrated in (a) of FIG. 2. The feature map is output after the convolution operation and the pooling operation are performed a plurality of times. Any network structure such as AlexNet, VGG-16, GoogLeNet, and Network in Network can be used as the feature extraction CNN. The size of the input image is set to have a width W, a height H, and the number of channels 3 (Red, Green, Blue). The size of the output feature map depends on the network structure used. For example, in the case of VGG-16, the width is W/16, the height is H/16, and the number of channels is 512. The following description will assume use of VGG-16 unless otherwise specified. Note that the VGG-16 used here has a configuration just before the fully connected layer, as will be described below with reference to FIG. 4. The VGG-16 including the fully connected layer will be described below in the classification CNN.

Next, the feature map is input to a candidate frame detection CNN. The candidate frame detection CNN is a three-layer CNN illustrated in (b) of FIG. 2, which includes an RPN frame variation map output convolutional layer and an RPN score map output convolutional layer. The RPN frame variation map and the RPN score map have a width W/16, a height H/16 each, and have the number of channels of 4×A and 2×A, respectively. A is the number of anchors, and the anchors represent the shape (aspect ratio, scale) of the candidate frame.

The position of the frame variation map and the score map in the spatial direction corresponds to the position of the original input image, and the maps have the frame variation of each of anchors (frame center movement amount and frame width expansion amount in each of x and y directions) and scores (polyp score and background score) in the channel direction. A coordinate value of the candidate frame and the RPN score representing the likelihood of polyp are calculated from the frame variation map and the score map, respectively.

Next, the feature map and the calculated coordinate values of the candidate frame are input to the ROI Pooling layer illustrated in (c) of FIG. 2 so as to perform cropping of the feature map for each of candidate frames and resizing using Max Pooling (subsampling of selecting a maximum value from 2×2 output of the previous layer). The output feature map for each of candidate frames has size of a width of 7, a height of 7, and 512 channels.

Next, the cropped feature map is input to the candidate frame classification Full Connect (FC) layer. The candidate frame classification FC layer is an FC layer including four layers illustrated in (d) of FIG. 2, and includes an FRCNN frame variation map output FC layer and an FRCNN score map output FC layer. The FRCNN frame variation map and the FRCNN score map have a horizontal width of 1, a vertical width of 1, and the number of cropped maps of M each, and have the number of channels of 4 (frame center movement amount and a frame width expansion amount in each of x and y directions)×A and 2 (polyp score and background score)×A, respectively. The final coordinate value of the detection frame and the FRCNN score indicating the likelihood of polyp are calculated similarly to the case of the candidate frame detection CNN.

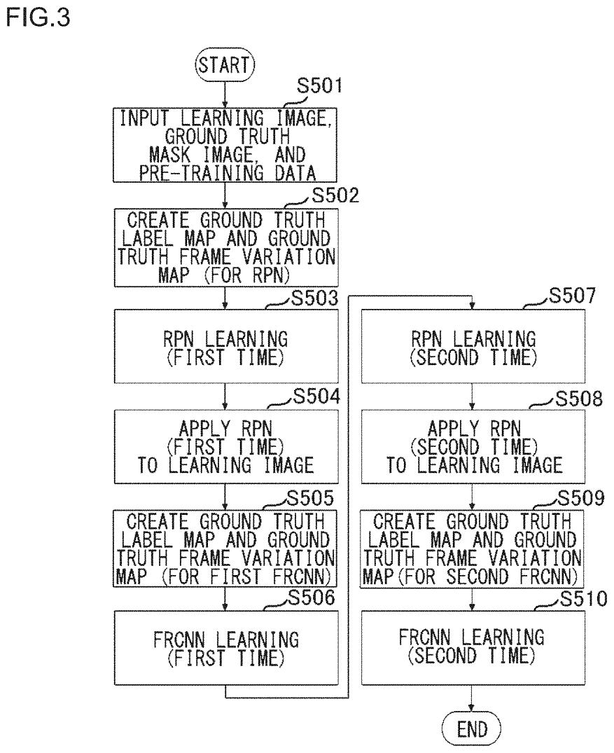

FIG. 3 illustrates a Faster R-CNN learning procedure. After learning is performed on RPN and FRCNN once for each, learning is performed one more time for RPN and FRCNN with the feature extraction CNN fixed so as to construct a network that shares the feature extraction CNN. First, in S501, a learning image and a correct mask image of a polyp as a detection target (an image in which a polyp region and a background region are separately colored) are input.

Next, in S502, a correct label map for RPN learning and a correct frame variation map are created from the correct mask image. The correct frame variation map and the correct label map have a width W/16 and a height H/16 each, and have the number of channels of 4 (frame center movement amount and frame width expansion amount in each of x and y directions)×A, and 1 (label)×A, respectively. For example, in a case where the overlapping rate between the coordinate value of the candidate frame corresponding to each of points on the map and the correct mask image is 50% or more, label=0 (polyp) will be stored in a correct label map; in a case where the overlapping rate is 0% or more and less than 50%, label=1 (background) will be stored in the correct label map. When label=0 (polyp), the variation obtained from the candidate frame to the rectangle circumscribing the polyp region of the correct mask image will be stored in the correct frame variation map.

Next, first RPN learning is performed in S503 based on the learning image, the correct label map, and the correct frame variation map that have been created. The optimization targets are both the feature extraction CNN and the candidate frame detection CNN. The Softmax cross entropies of the correct label map and the RPN score map are added to the weighted Smooth L1 Loss of the correct frame variation map and the frame variation map, thus defined as the loss function. Stochastic Gradient Descent (SGD) is used for optimization.

Next, in S504, the constructed RPN is applied to the learning image to calculate a polyp candidate frame and an RPN score representing the likelihood of polyp. Subsequently, in S505, a correct label map and a correct frame variation map for FRCNN learning are created from the detected candidate frame and correct frame mask image. The correct frame variation map and the correct label map have a width W/16, a height H/16, and the number of output candidate frames M each, and have the number of channels of 4 (frame center movement amount and frame width expansion amount in each of x and y directions)×A, and 1 (label)×A, respectively. For example, in a case where the overlapping rate between the coordinate value of the detected candidate frame and the correct mask image is 50% or more, label=0 (polyp) will be selected; in a case where the overlapping rate is more than 0% and less than 50%, label=1 (background) will be selected. When label=0 (polyp), the variation obtained from the candidate frame to the rectangle circumscribing the polyp region of the correct mask image will be stored in the correct frame variation map.

Next, first FRCNN learning is performed in S506 based on the learning image, the correct label map, and the correct frame variation map that have been created. The optimization targets are both the feature extraction CNN and the candidate frame classification FC layer. The loss function and optimization method same as RPN will be used.

Next, second RPN learning is performed in S507 based on the correct label map and the correct frame variation map used in the first RPN learning. The feature extraction CNN is fixed by the learning result of the first FRCNN, and the candidate frame detection CNN alone will be used as the optimization target.

Next, in S508, the trained RPN is applied to the learning image to calculate a polyp candidate frame and an RPN score representing the likelihood of polyp. Subsequently, in S509, a correct label map and a correct frame variation map for FRCNN learning are created from the detected candidate frame and the correct frame data similarly to the first time.

Finally, second FRCNN learning is performed in S510 based on the learning image, the correct label map, and the correct frame variation map that have been created. The feature extraction CNN is fixed by the learning result of the first FRCNN, and the candidate frame classification FC layer alone will be used as the optimization target.

The polyp detection process has been described above using an example of the Faster R-CNN.

Next, the classification (discrimination) process performed by a CNN will be described. First, a classification CNN is trained using benign and malignant polyps. Next, when a polyp region is detected, the region is input to the classification CNN and whether the region is a benign polyp or a malignant polyp is discriminated. The classification is not limited to two categories of benign or malignant. For example, in the NICE classification of colorectal polyps, polyps are divided into Type1, Type2, and Type3 in the order of benign to malignant.

The classification CNN for discriminating the malignancy of polyps will be described below.

FIG. 4 illustrates a schematic processing configuration of the CNN. The reference sign 605 is a CNN, 605-C is a convolution layer, 605-P is a pooling layer, 605-FC is a fully connected layer, and 605-D is an endoscopic image database (DB). Although FIG. 4 illustrates an example in which the convolution layer 605-C and the pooling layer 605-P are repeated three times, the number of times of iterations is not particularly limited. In addition, while this is an example in which the fully connected layer 605-FC includes two layers, the number of layers is not particularly limited. Moreover, the convolution layer include a process of applying a nonlinear function (ReLU) after the convolution process.

Here, an example using VGG-16 as the CNN will be described.

VGG-16 uses a 3×3 convolution filter to apply a convolution result with the input image to the nonlinear function ReLU. MaxPooling is used after two or three convolution layers used in a row. VGG-16 uses 13 convolution layers and 5 times of MaxPooling, and finally is connected to three fully connected layers.

Next, the CNN learning method will be described. First, the training data for the gastrointestinal endoscope is prepared. For example, Type1, Type2, Type3, or the like, of the NICE classification are labeled to images, and a set of images and labels is referred to as a training dataset. Here, an NBI image or a normal light image is used as the image.

When the number of this training data set is about tens of thousands, it is allowable to directly train the VGG-16 network. However, when the number is less than that, it is also allowable to use a pre-trained VGG-16 network trained with the large-scale image DB such as ImageNet to apply fine-tuning (type of transfer learning) using the gastrointestinal endoscope image dataset.

An image is input, and the convolution and pooling results propagate as signals. The difference between an output layer signal and a training signal based on a label corresponding to the input image is calculated. This difference as an error propagates in the opposite direction, and the weight of each of layers is updated by using the above-described stochastic gradient descent (SGD) or the like to decrease the error. When learning is completed, the weight of each of layers is fixed.

When an unknown image is input during the test, the signal propagates through the CNN, and the image is classified based on the signal value output at the output layer. For example, in the NICE classification of polyps, the label that outputs the maximum value of the output signals of Type1, Type2, and Type3 is determined as an estimation result.

The processing of the classification CNN has been described above.

While this is an example in which detection CNNs and classification CNNs are separately prepared, it is allowable to employ a configuration in which detection and classification are performed simultaneously. As in the Faster R-CNN detection and classification (discrimination) using one network is proposed, it is allowable to employ such a configuration. In this case, a classification process of classifying whether the lesion indicated by the region of interest is benign or malignant is to be executed before determining whether the region of interest is a diagnostically inadequate region.

Next, operations of the image diagnosis support system 100 configured as above will be described.

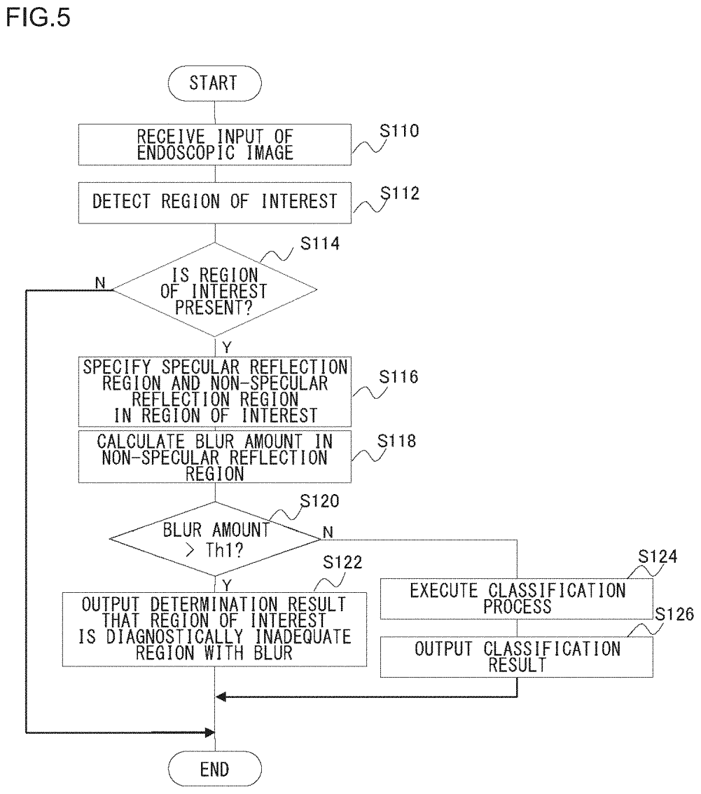

FIG. 5 is a flowchart illustrating an example of a series of processes in the image diagnosis support system 100. The image input unit 110 receives input of an endoscopic image (S110). The region of interest detector 112 executes a detection process of detecting a region of interest on an endoscopic image (S112). When the region of interest is detected, that is, when the region of interest exists in the endoscopic image (Y in S114), the specular reflection region and the non-specular reflection region in the region of interest are specified (S116). The blur amount calculation unit 116 calculates the blur amount in the non-specular reflection region (S118). The determination unit 118 determines whether the blur amount is larger than a threshold Th1, that is, whether the region of interest is a diagnostically inadequate region with a blur (S120). When the blur amount is larger than the threshold Th1, that is, when the region of interest is a diagnostically inadequate region with a blur (Y in S120), classification processing would not be executed, and the output unit 122 outputs a determination result that the region of interest is a diagnostically inadequate region with a blur (S122). In a case where the blur amount is the threshold Th1 or less, that is, the region of interest is not a diagnostically inadequate region (N of S120), the classifier 120 executes a classification process on the lesion indicated by the region of interest (S124). The output unit 122 outputs the result of the classification process (S126). In a case where no region of interest is detected (N in S114), S116 to S126 are skipped and the process ends.

According to the image diagnosis support system 100 of the first embodiment described above, when the non-specular reflection region of the region of interest is blurred, it is determined that the region of interest is an inadequate region that is inadequate for diagnosis. With this configuration, even when the image is an endoscopic image having a blur or the like in a region unrelated to the region of interest, the endoscopic image is determined as a diagnosis target.

### Second Embodiment

FIG. 6 is a block diagram illustrating the functions and configuration of an image diagnosis support system 200 according to a second embodiment. FIG. 6 corresponds to FIG. 1 of the first embodiment. The main difference from the first embodiment is that it is determined whether the region of interest has a shake instead of a blur, and it is determined that the region of interest is a diagnostically inadequate region in a case where the region of interest has a shake. Hereinafter, differences from the image diagnosis support system 100 according to the first embodiment will be mainly described.

The image diagnosis support system 200 includes an image input unit 110, a region of interest detector 112, a specifying unit 114, a circularity calculation unit 216, a determination unit 218, a classifier 120, and an output unit 122.

The circularity calculation unit 216 first performs a connection process on the specular reflection region of the region of interest. The connection process is to perform a labeling process regarding a continuous specular reflection region as one block.

Subsequently, the circularity calculation unit 216 calculates a circularity (C) of each of connected regions by the following Formula (2).

$$\begin{matrix}Formula ( 2 ) & \\ C = 4 \frac{1}{π} \frac{S}{{L}^{2}} & Formula ( 2 )\end{matrix}$$

Here

S: Area of specular reflection region

L: Perimeter of specular reflection region

The circularity calculation unit 216 subsequently defines the maximum circularity of the circularity of each of connected regions as the circularity of the specular reflection region.

The determination unit 218 of the present embodiment determines whether the region of interest is a diagnostically inadequate region with a shake based on the image processing result for the specular reflection region of the region of interest, that is, based on the circularity of the specular reflection region. Here, the specular reflection region normally has a shape close to a circle when it has no shakes, leading to a circularity close to 1, whereas when it has a shake, the shape is close to an ellipse or a line segment, leading to a circularity smaller than 1. Therefore, a value less than 1 is set as a threshold Th2 for determining whether the region includes a shake. The determination unit 218 determines that the region of interest has a shake, that is, the region of interest is determined as a diagnostically inadequate region when the circularity of the specular reflection region is less than the threshold Th2, and determines that the region of interest has no shakes, that is, the region of interest is not a diagnostically inadequate region when the circularity of the specular reflection region is the threshold Th2 or more.

Operations of the image diagnosis support system 200 according to the second embodiment will be described.

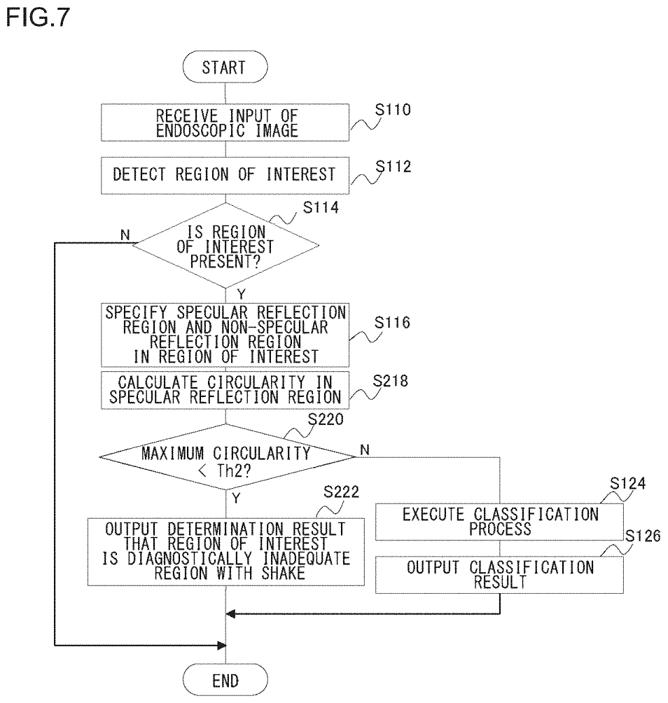

FIG. 7 is a flowchart illustrating an example of a series of processes in the image diagnosis support system 200. The differences from FIG. 5 will be mainly described. The circularity calculation unit 216 calculates the circularity of the specular reflection region (S218). The determination unit 218 determines whether the circularity is less than the threshold Th2, that is, whether the region of interest is a diagnostically inadequate region with a shake (S220). In a case where the circularity is less than the threshold Th2, that is, the region of interest is a diagnostically inadequate region with a shake (Y of S220), the classification process would not be executed, and the output unit 122 will output a determination result that the region of interest is a diagnostically inadequate region with a shake (S222). In a case where the circularity is the threshold Th2 or more, that is, the region of interest is not a diagnostically inadequate region (N of S220), the classifier 120 executes a classification process on the lesion indicated by the region of interest (S124). The output unit 122 outputs the result of the classification process (S126).

According to the image diagnosis support system 200 of the second embodiment described above, the region of interest is determined as an inadequate region that is inadequate for diagnosis in a case where the specular reflection region of the region of interest has a shake. With this configuration, even when the image is an endoscopic image having a shake or the like in a region unrelated to the region of interest, the endoscopic image is determined as a diagnosis target.

### Third Embodiment

FIG. 8 is a block diagram illustrating the functions and configuration of an image diagnosis support system 300 according to a third embodiment. FIG. 8 corresponds to FIG. 1 of the first embodiment. The main difference from the first embodiment is that it is determined whether the region of interest has a shake instead of a blur, and it is determined that the region of interest is a diagnostically inadequate region in a case where the region of interest has a shake. Hereinafter, differences from the image diagnosis support system 100 according to the first embodiment will be mainly described.

The image diagnosis support system 300 includes an image input unit 110, a region of interest detector 112, a specifying unit 114, a direction frequency analyzer 316, a determination unit 318, a classifier 120, and an output unit 122.

The direction frequency analyzer 316 first extracts edges from the specular reflection region and the non-specular reflection region individually in the region of interest. Subsequently, the direction frequency analyzer 316 extracts a line segment from the extracted edge. Extraction of the line segment may use a known technique such as Hough transform. Extraction of edges and line segments may also use the technique of the Line Segment Detector.

The direction frequency analyzer 316 analyzes the extracted directional line segment based on the direction. Specifically, for each of the specular reflection region and the non-specular reflection region, the direction frequency analyzer 316 classifies each of the extracted directional line segments into angular ranges obtained by dividing 180 degrees by M equal parts at θ degrees intervals (for example, dividing 180 degrees by 12 equal parts at 15 degrees intervals) and then accumulates the length of the directional line segment for each of angular ranges to create a histogram (frequency distribution) of the directional line segments. The direction frequency analyzer 316 sets the angular range having the largest histogram value as a main direction of the directional line segment individually for the specular reflection region and the non-specular reflection region.

The determination unit 318 determines that the region of interest has a shake, that is, the region of interest is a diagnostically inadequate region when the main direction of the directional line segment of the specular reflection region and the main direction of the directional line segment of the non-specular reflection region match, and determines that the region of interest has no shake, that is, the region of interest is not a diagnostically inadequate region when there is no match. In consideration of an error, the determination unit 318 may determine that main directions match also in a case where the angular range being the main direction of the directional line segment of the specular reflection region and the angular range being the main direction of the directional line segment of the non-specular reflection region are adjacent to each other.

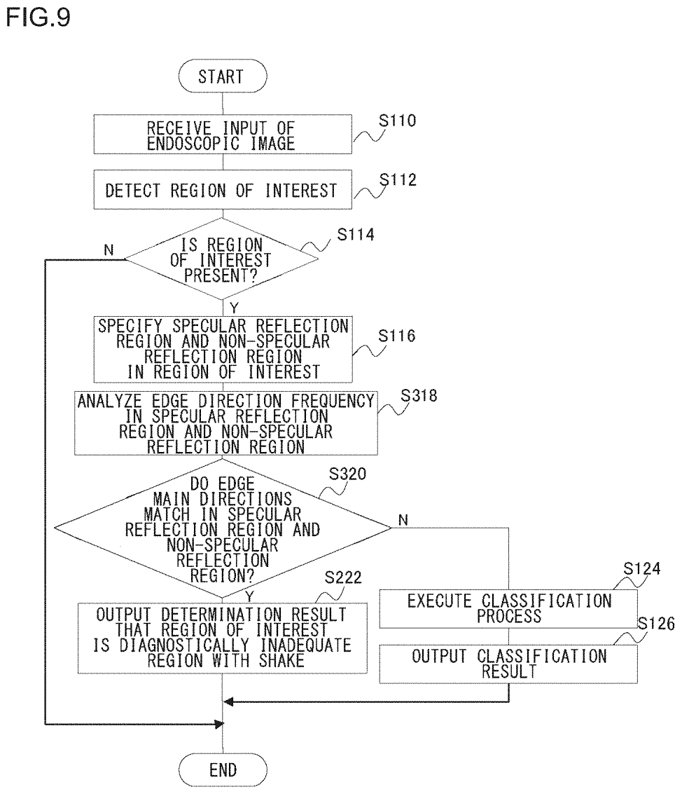

Operations of the image diagnosis support system 300 according to the third embodiment will be described. FIG. 9 is a flowchart illustrating an example of a series of processes in the image diagnosis support system 300. The differences from FIG. 5 will be mainly described. The direction frequency analyzer 316 extracts an edge individually from the specular reflection region and the non-specular reflection region of the region of interest and then specifies the main direction of each of edges of the specular reflection region and the non-specular reflection region (S318). The determination unit 318 determines whether the main direction of the specular reflection region and the main direction of the non-specular reflection region match, that is, whether the region of interest is a diagnostically inadequate region with a shake (S320). In a case where the main directions match, that is, the region of interest is a diagnostically inadequate region with a shake (Y of S320), the classification process will not be executed, and the output unit 122 will output a determination result that the region of interest is a diagnostically inadequate region with a shake (S222). When the main directions do not match, that is, the region of interest is not a diagnostically inadequate region (N in S320), the classifier 120 performs the classification process on the lesion indicated by the region of interest (S124). The output unit 122 outputs the result of the classification process (S126).

According to the image diagnosis support system 300 of the third embodiment described above, the region of interest is determined as an inadequate region that is inadequate for diagnosis in a case where the specular reflection region of the region of interest has a shake. With this configuration, even when the image is an endoscopic image having a shake or the like in a region unrelated to the region of interest, the endoscopic image is determined as a diagnosis target.

The present invention has been described with reference to the embodiments. The present embodiment has been described merely for exemplary purposes. Rather, it can be readily conceived by those skilled in the art that various modification examples may be made by making various combinations of the above-described components or processes, which are also encompassed in the technical scope of the present invention.

### First Modification

The embodiments are the cases where the image diagnosis support system 100 supports diagnosis of a lesion using an endoscopic image captured by a medical endoscope. However, the present invention is not limited to this. The image diagnosis support system 100 can also be applied to cases of supporting flaw inspection of a metal surface using an endoscopic image captured by an industrial endoscope. For example, in order to verify the degree of damage for a scratch, it is allowable to detect a region of interest, which is a scratch candidate region, from an endoscopic image, specifying a specular reflection region and a non-specular reflection region among the region of interest, extracting an edge from the non-specular reflection region, calculating a blur amount of the edge, determining whether the region of interest is a diagnostically inadequate region with a blur based on the blur amount, and outputting a classification result obtained by execution of classification process of classifying the damage degree of the scratch when it is not a diagnostically inadequate region, or outputting a result that the region of interest is a diagnostically inadequate region without executing the classification process.

### Second Modification

The methods of the first to third embodiments may be flexibly combined to determine whether the region of interest is a diagnostically inadequate region.

For example, any two methods of the methods of the first to third embodiments may be combined. In this case, a region of interest may be determined as a diagnostically inadequate region in a case where the region of interest is determined as a diagnostically inadequate region by at least one method; or a region of interest may be determined as a diagnostically inadequate region in a case where the region of interest is determined as diagnostically inadequate by the two methods.

Furthermore, all the methods of the first to third embodiments may be combined with each other, for example. In this case, a region of interest may be determined as a diagnostically inadequate region in a case where the region of interest is determined as a diagnostically inadequate region by at least one method; a region of interest may be determined as a diagnostically inadequate region in a case where the region of interest is determined as a diagnostically inadequate region by two or more methods; or a region of interest may be determined as a diagnostically inadequate region in a case where the region of interest is determined as a diagnostically inadequate region by the three methods.

### Third Modification

It is allowable to determine whether the region of interest is a diagnostically inadequate region by first calculating a blur amount and a shake amount in the region of interest as features and then making evaluation using a combination of these features. Examples of the shake amount include the circularity of the second embodiment, the variance calculated from the histogram of the third embodiment, the main direction matching degree calculated from the main direction of the directional line segment of the third embodiment.

In addition, it is allowable to use a learning or identification system by using a support vector machine (SVM) with the above-described features as vector components.

### Fourth Modification

In the embodiment, the case where the image diagnosis support system 100 includes the classifier 120 has been described. However, the present invention is not limited to this, and it is conceivable to employ a configuration that includes no classifier 120. In this case, a radiologist determines whether the lesion indicated by the region of interest is benign or malignant. In a case where the region of interest is a diagnostically inadequate region, the output unit 122 may display the determination to the radiologist.

What is claimed is:

1. An image diagnosis support system comprising a processor that includes hardware,
wherein the processor is configured to:
receive an input of an image,
specify a specular reflection region and a non-specular reflection region in a region of interest in the image, and
determine whether the region of interest is an inadequate region that is inadequate for diagnosis on the basis of an image processing result for at least one of the specular reflection region and the non-specular reflection region.

2. The image diagnosis support system according to claim 1,
wherein the processor is configured to determine whether the region of interest is an inadequate region with a blur on the basis of the image processing result for the non-specular reflection region.

3. The image diagnosis support system according to claim 2,
wherein the processor is configured to:
calculate a blur amount of the non-specular reflection region, and
determine whether the region of interest is an inadequate region with a blur on the basis of the calculated blur amount.

4. The image diagnosis support system according to claim 3,
wherein the processor is configured to calculate the blur amount using the image before applying a Gaussian filter and the image after applying the Gaussian filter.

5. The image diagnosis support system according to claim 1,
wherein the processor is configured to determine whether the region of interest is an inadequate region with a shake on the basis of the image processing result for the specular reflection region.

6. The image diagnosis support system according to claim 5,
wherein the processor is configured to:
calculate circularity of the specular reflection region of the specular reflection region, and
determine whether the region of interest is an inadequate region with a shake on the basis of the calculated circularity.

7. The image diagnosis support system according to claim 1,
wherein the processor is configured to determine that the region of interest is an inadequate region with a shake in a case where a first direction specified based on an edge detected by image processing on the specular reflection region matches a second direction specified based on an edge detected by image processing on the non-specular reflection region.

8. The image diagnosis support system according to claim 1,
wherein the processor is configured to classify the region of interest based on a feature of the region.

9. The image diagnosis support system according to claim 8,
wherein the processor is configured to:
classify the region of interest based on the feature of the region in a case where determination has been made that the region of interest is not an inadequate region, and
output a classification result of the region of interest.

10. The image diagnosis support system according to claim 8,
wherein the processor is configured to output a classification result of the region of interest in a case where determination has been made that the region of interest is not a diagnostically inadequate region.

11. The image diagnosis support system according to claim 8,
wherein the region of interest is a lesion candidate region in the image, and
wherein the processor is configured to classify malignancy of the lesion candidate region.

12. The image diagnosis support system according to claim 8,
wherein the processor is configured to execute a classification process by using a convolutional neural network.

13. An image diagnosis support method comprising:
receiving an input of an image; and
determining whether a region of interest is an inadequate region that is inadequate for diagnosis on the basis of an image processing result for at least one of a specular reflection region and a non-specular reflection region in the region of interest in the image.

14. A non-transitory computer readable medium encoded with a program executable by a compute, the program comprising:
receiving an input of an image; and
determining whether a region of interest is an inadequate region that is inadequate for diagnosis on the basis of an image processing result for at least one of a specular reflection region and a non-specular reflection region in the region of interest in the image.
